(19) United States
(12) Patent Application Publication
(10) Pub. No.: US 2008/0208001 A1
(43) Pub. Date: Aug. 28, 2008

(54) CONFORMING ENDOSCOPE

(76) Inventor: Ron HADANI, Cresskill, NJ (US)

Correspondence Address: MORRISON & FOERSTER LLP, 755 PAGE MILL RD, PALO ALTO, CA, 94304-1018, US

(21) Appl. No.: 12/035,337

(22) Filed: Feb. 21, 2008

Related U.S. Application Data
(60) Provisional application No. 60/903,798, filed on Feb. 26, 2007.

(51) Int. Cl. A61B 1/008

(52) U.S. Cl. 600/128; 600/135; 600/141

(57) ABSTRACT

Described herein are endoscopes having outer perimeter shapes configured to conform to the particular anatomy through which they will navigate. The outer perimeter cross-section of the endoscope may have one or more regions (e.g., longitudinal protuberances, edges, or narrow regions) that may allow the endoscope to conform with or fit within narrow or irregularly shaped regions of a body lumen, channel or passageway. Thus, the cross-sectional shape of the insertion tube of the endoscope may have an outer perimeter cross-section that is substantially D-shaped, oval, triangular, lobular, teardrop shaped, or the like. These non-circular cross-sectional shapes are matched approximately to the geometry of the region of the body into which the endoscope will be inserted, thereby allowing the endoscope to more precisely fit within a body lumen.

### CROSS REFERENCE TO RELATED APPLICATIONS

This application claims priority benefit under 35 U.S.C. § 119(e) to U.S. Provisional Patent Application Ser. No. 60/903,798, filed Feb. 26, 2007, the disclosure of which is hereby incorporated by reference herein in its entirety.

### FIELD

The devices described here relate to the field of endoscopy. More specifically, the devices relate to endoscopes capable of accommodating specific anatomies.

### BACKGROUND

Endoscopes are generally used to examine various body organs. For example, endoscopes are widely used to view the esophagus, stomach, small and large bowel, bile ducts, large and small respiratory airways, nasal cavities, urethras, and fallopian tubes. Endoscopy has been increasingly used not only to diagnose, but also to treat a growing number of medical conditions by employing attachments that can excise tissue (e.g., polyps and small tumors), remove foreign objects, and/or provide cautery, coagulation, or other hemostatic functions. Although the overall rate of complications is assumed to be low, the occurrence of complications is highly dependent on the experience and skill of the endoscopist.

During an endoscopic procedure, the endoscope is typically maneuvered through a lumen, channel, orifice or passageway of the organ of interest. The viewing of images is enabled by a small video camera in the endoscope distal tip that can capture the images for display, or by a fiberoptic bundle that can transfer the images to an eyepiece for immediate viewing or for capture (e.g., by a camera). Navigation of the endoscope through the lumen, channel, orifice or passageway is oftentimes difficult due to the nature of the particular anatomy being examined. For example, advancement of the endoscope may be impeded by the curvature or tortuosity of luminal organs or by the presence of other anatomical structures that form obstacles in the endoscope path. In these cases, excessive or repeated force may be used in maneuvering the endoscope, which increases the risk of tissue trauma and/or tissue perforation. Although flexible and steerable endoscopes have been developed as an attempt to lower the risk of trauma and perforation, endoscopic access still poses a significant challenge.

Commercially available endoscopes typically have a substantially circular cross-section when they are inserted into a subject's anatomy. Although endoscopes having partial non-circular cross-sections have been described, the insertion portion of these endoscopes are used with a sheath or jacket so that the device or system presents a substantially circular cross-section to the body when the insertion portion is actually inserted into a subject's body. For example, U.S. Pat. No. 5,271,381 to Ailinger et al., describes an endoscope having D-shaped vertebrae. When the insertion tube of the Ailinger endoscope is used to perform a medical procedure on a patient, a sheath incorporating a channel is positioned around the insertion tube so that the outer perimeter of the device inserted into the patient is substantially circular. Thus the outer surface of the endoscope that contacts the walls of the body lumen or passageway (i.e., the body-contacting surface) has a substantially round cross-section.

Other examples of endoscopes having regions of non-circular cross-section include U.S. Pat. No. 4,947,827, U.S. Pat. No. 5,213,093, U.S. Pat. No. 5,199,417, GB 2068139, U.S. Pat. No. 4,869,238, US RE 34,110, and U.S. Pat. No. 4,607,622. Similar to the Ailinger et al. device, most of these endoscopes have internal structures that are non-circular (e.g., non-circular vertebra), but the outer diameter of the endoscope that contacts the patient during use is typically circular, or symmetrical. Endoscopes having circular outer surfaces may be limited in their ability to interact with internal body structures, particularly in non-circular body lumen.

Accordingly, it would be desirable to have endoscopes that more accurately conform to the shape of a body lumen. It would also be desirable to have endoscopes capable of accommodating the geometry of the orifice, lumen, organ, or passageway being navigated. Similarly, it would be desirable to have endoscopes that employ their enhanced maneuverability in the evaluation and treatment of various medical conditions.

### SUMMARY

Described here are endoscopes that are configured to conform to the particular anatomy, e.g., lumen, channel, or passageway, being navigated. In some of the devices described herein, the outer circumferential (or perimeter) cross-section of the endoscope is shaped (e.g., having protuberances, edges, or narrow regions) to allow the endoscope to conform with or fit within narrow or irregularly shaped regions of a body orifice, lumen, channel or passageway. For example, the cross-sectional shape of the insertion tube of the endoscope (the portion that is inserted into the body) may have an outer perimeter cross-section that is substantially D-shaped, oval, triangular, lobular, teardrop shaped, or the like. In some variations, the outer perimeter cross-section may have more than one protuberance (e.g., “fingers”). In some variations, the outer perimeter cross-section is asymmetric. In some variations, the cross-section is substantially triangular. These outer cross-sectional shapes may allow the endoscope to more precisely fit within a body lumen, particularly when the outer cross-sectional shape of the endoscope matches the inner cross-sectional shape of at least a portion of the body lumen or orifice into which it is inserted (e.g., a nasal passage, urethra, etc.).

The endoscopes described herein may be part of a system, including a system for performing a diagnostic or therapeutic procedure. An endoscope system may include an endoscope (including any of the endoscopes described herein), and a sheath forming an outer surface of the endoscope system. The sheath may conform to the shape (e.g., the non-circular shape) of the endoscope cross-section. In some variations, the sheath forms the non-circular outer perimeter cross-section of the endoscope.

An endoscope for performing a diagnostic or therapeutic procedure may include an elongate portion that is non-round in cross-section (e.g., non-circular), for insertion into an organ or passageway of the body, and a bending portion. The cross-section of the elongate portion may have a major axis and a minor axis, and the elongate portion may be configured to accommodate the geometry of the organ or passageway. The bending portion may include a plurality of vertebrae. The elongate portion may be asymmetric about the minor axis, the major axis, or both the minor and major axis. For example, the cross-section though the elongate portion may be D-shaped, teardrop-shaped, or the like. The elongate portion and the bending portion may make up the insertion portion (insertion tube).

In some variations, the vertebrae within the endoscope are non-round in cross-section. Thus, the vertebrae may have approximately the same cross-sectional shape as the outer cross-section of the insertion portion of the endoscope. For example, the cross-sectional shape of the vertebrae may be D-shaped in cross-section. In some variations, the elongate portion has the same outer cross-sectional shape as the bending portion. For example, the elongate portion may be D-shaped in cross-section and the bending portion may be D-shaped in cross-section. In some variations, the elongate portion has a different outer perimeter cross-section than the bending portion. The bending portion may be located at the distal end of the endoscope, and the elongate portion may be located proximal to the bending portion.

The endoscopes and endoscope systems described herein may be used in any appropriate body region (e.g., organ, lumen, orifice, passageway, etc.). Thus, the devices described herein may include an outer surface or region that is configured to conform to an inner surface of the body region (e.g. within a lumen of the organ). In particular, the endoscopes described herein may conform to body lumens having non-circular cross-sections, including body lumens having ridges, narrowings, folds, or canals. For example, the devices described herein may be used within a urologic organ (e.g., a ureter, urethra, etc.), a respiratory passageway (e.g., a nasal passageway), or the like.

The endoscope (or a system including an endoscope) may include a sheath. A sheath may have a proximal end, a distal end, and a sheath lumen extending from the proximal end to the distal end configured for slidable advancement of the elongate portion. The sheath may have variable stiffness between the proximal end and distal end. In some variations, the sheath lumen may be non-round in cross-section. For example, the sheath lumen may be D-shaped in cross-section. In some variations, the outer cross-section of the insertion portion of an endoscope system is shaped into the non-circular cross section (e.g., an oval, triangular, teardrop, D-shape, etc.) by the sheath.

In some variations, the elongate portion of the endoscope includes a shaft having a proximal end, a distal end, and a length therebetween of variable stiffness. As mentioned, the bending portion may be part of the elongate portion. In some variations, the bending portion rotates about a minor axis of the cross-section through the device. In some variations, the bending portion rotates about a major axis. In some variations, the length of the minor axis is about 3 mm or less. In some variations, the length of a major axis is about 4.3 mm or less.

Also described herein are endoscopes for performing a diagnostic or therapeutic procedure that include an elongate portion (non-round in cross-section), for insertion into a portion of a body, and a bending portion. The cross-section of the elongate portion and/or the bending portion may have a major axis and a minor axis. The elongate portion may be configured to accommodate the geometry of the portion of a body. The bending portion may include a plurality of vertebrae.

Also described herein are endoscopes for performing a diagnostic or therapeutic procedure that include an elongate portion with a non-round outer perimeter (in cross-section), for insertion into an organ or passageway of the body, and a bending portion. The outer perimeter cross-section may have one or more protuberances. The protuberance(s) may form an edge or ridge along the length of the insertion portion of the endoscope. The protuberance may be configured to conform to the geometry of an organ or passageway, and the bending portion comprises a plurality of vertebrae.

Also described herein are endoscope for performing a diagnostic or therapeutic procedure comprising an insertion portion having a non-circular outer perimeter in cross-section (insertable into an organ or passageway of the body while maintaining the non-circular outer perimeter), and a bending portion. The outer perimeter cross-section may include a protuberance, configured to conform to the geometry of the organ or passageway. The bending portion typically comprises a plurality of vertebrae. The insertion portion is configured so that the outer surface of the insertion portion is the body-contacting surface, and this surface has an outer perimeter cross-section that is non-circular. There are many ways that the insertion portion may be configured to have an outer surface that contacts a subject's body while maintaining the non-circular outer perimeter cross-section. For example, the outer surface may comprise a biocompatible material (possibly including a lubricant). The outer surface may be smooth, or substantially continuous so that it does not harm the body tissue. Furthermore, the insertion portion may be sufficiently rigid to maintain the non-circular outer perimeter cross-section when inserted into a subject.

In some variations, the outer perimeter cross-section of the endoscopes described herein has a different outer perimeter cross-section along the length of the insertion portion. For example, the outer perimeter cross-section may change across the length of the insertion portion (e.g., from the proximal end to the distal end). In some variations, the outer perimeter cross-section of the insertion portion is larger towards the proximal end of the insertion portion than the distal end of the insertion portion.

Also described herein are systems for performing a diagnostic or therapeutic procedure, including an endoscope having an elongate portion (non-round in cross-section), for insertion into an organ or passageway of the body and a bending portion, and an outer sheath configured to fit over the endoscope. The outer perimeter of the cross-section through the endoscope and the outer sheath has a non-round cross-section. The cross-section of the elongate portion may have a major axis and a minor axis. The elongate portion may be configured to accommodate the geometry of the organ or passageway. The bending portion may include a plurality of vertebrae.

Also described herein are methods of performing a diagnostic or therapeutic procedure using any of the devices or systems described herein. For example, a method of performing a diagnostic or therapeutic procedure may include inserting the insertion portion of an endoscope system into a body lumen, wherein the insertion portion of the endoscope system has a non-circular outer diameter in cross-section, and wherein the inner diameter of the body lumen has a non-circular cross-section. The non-circular outer diameter cross-section may be a D-shaped cross-section. The non-circular outer diameter cross-section may comprise an asymmetric cross-section.

The method may also include preparing the insertion portion of the endoscope system by placing a sheath over the endoscope prior to inserting the insertion portion of the endoscope into the body lumen.

### BRIEF DESCRIPTION OF THE DRAWINGS

FIGS. 1A and 1B show a perspective and cross-sectional view (respectively) of a prior art endoscope.

FIG. 2 is a perspective view of a conforming endoscope having a handle, insertion tube, and bending section.

FIG. 3A is an expanded view of the insertion tube and bending section shown in FIG. 2.

FIG. 3B is a cross-sectional view of through the bending section shown in FIG. 3A.

FIGS. 4A-4C are expanded views of the bending section and vertebrae shown in FIG. 3A.

FIG. 5A shows a cross-section through an outer perimeter profile of one variation of a conforming endoscope as described herein.

FIGS. 5B-5F are cross-sections showing the outer perimeter profile through different conforming endoscopes as described herein.

FIGS. 6A and 6B are cross-sections through the insertion regions of variations of conformable endoscopes as described herein.

### DETAILED DESCRIPTION

Conforming endoscopes may be inserted into any appropriate body cavity, orifice, lumen, passageway, or the like in order to visualize or treat the body. The conforming endoscopes described herein typically include an elongate insertion tube (or insertion region) that is configured to be inserted into a subject's body. The insertion tube is configured to accommodate the geometry of the portion of the body into which the endoscope will be inserted. As described below, the non-circular outer cross-section may allow at least a portion of the insertion tube to better fit within the shape of the body lumen or cavity, since many regions of the body have lumens with internal cross-sections that are non-circular.

The insertion tube of the endoscopes described herein generally includes a bending section near the distal tip so that the orientation and position of the distal tip is steerable by a user (e.g., physician). An endoscope may also include a handle connected (or connectable) to the insertion tube. The bending section of the endoscope must be sufficiently controllable and flexible that the physician can position the tip in any necessary location or orientation within the body cavity being examined. For most endoscopes, the tip is able to perform a 90° or greater turn in an up, down, left, or right direction. In some types of endoscopes, the tip turns only in an up or down direction to provide the control required by the user. In other types of endoscopes, the tip may optionally be manipulated up and down as well as left and right.

Although many of the examples of endoscopes and insertion tubes included herein describe flexible (e.g., bendable or steerable) endoscopes, the endoscopes may also be configured as stiff or rigid endoscopes. For example, a rigid endoscope may have an insertion section that has a non-circular outer perimeter cross-section along its length, as described in more detail herein.

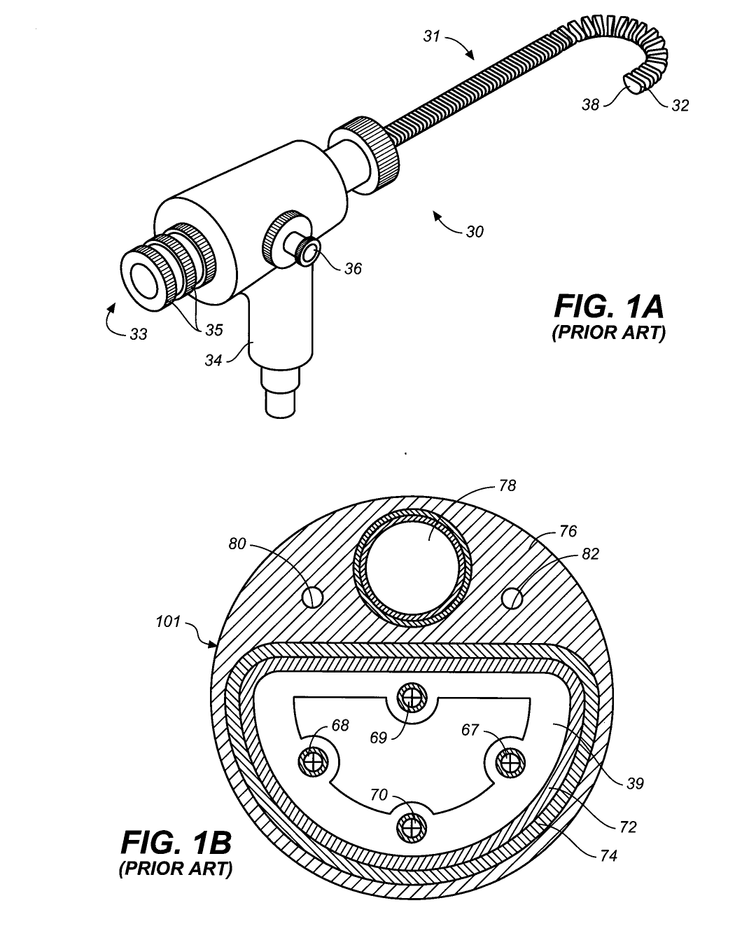

As mentioned, the insertion tubes of most prior art endoscopes (or endoscope systems) have a substantially circular outer cross-section. This means that the outer surface of the insertion tube that can contact the subject is typically circular in cross-section. For example, FIG. 1A shows a perspective view of a prior art endoscope, and FIG. 1B shows a cross-sectional view of through the insertion tube of the same endoscope. FIGS. 1A and 1B are taken from U.S. Pat. No. 5,271,381.

In the prior art endoscope 30 shown in FIG. 1A, an insertion tube 31 includes a bending section 32, and a handle 34 having control knobs (or levers) 36 which remain outside the body and are manipulated by an operator to articulate the bending section 32 of the insertion tube 31. This device also includes an eyepiece 33 having diopter adjustment ring(s) 35 that permit the operator to view objects near the tip of the endoscope. The insertion tube 31 is sufficiently flexible to permit it to travel through the interior of the body within a selected body channel or orifice, such as the nasal cavity, esophagus, the urinary tract, the large intestine, or the like. The insertion tube region shown in FIG. 1A is generally sheathed before being inserted into a body orifice or lumen, so that the outer surface of the insertion tube is not the exposed metal or braided region shown in FIG. 1A, but has a cross-section as shown in FIG. 1B.

In FIG. 1A, the bending section 32 of the insertion tube 31 is steerable by the operator. In general, an endoscope may be steered by an operator retracting or extending control wires through manipulation of knob(s) or lever(s) 36 to move the bending section 32 up, down, left or right during the advancement of the insertion tube to the desired location in the body, as well as after advancement to the desired location for the purpose of viewing the area of interest. Steering of the bending section 32 permits the operator to place the tip 38 at a selected location within the body. The operator may then view a portion of the body through a fiber-optic image bundle, (or a video chip located at the distal tip) remove a sample for biopsy purposes, place a chemical at the selected location, or perform other medical procedures. The bending section 32 must be sufficiently flexible to bend through 90° or more to perform various medical procedures. To permit this controlled bending, the bending section 32 may be comprised of a plurality of individual vertebrae which pivot with respect to each other.

FIG. 1B shows a cross-section through the insertion tube of the prior art device of FIG. 1A. The outer perimeter 101 of the cross-section through the insertion tube is substantially circular. Thus, the shape of the insertion tube that may contact a subject's body (e.g., an orifice, lumen, etc.) is an elongate tube having a circular cross-section. In FIG. 1B, a sheath 76 is attached over the inner portion of the insertion tube. The bending section 32 is composed of a plurality of individual vertebrae 39 held together by control wires 67-70 extending through respective apertures 60-63. A metal braid 72 is wrapped around the assembled vertebra 39 to form the bending section 32 of the insertion tube 31. A polymeric jacket 74 is then wrapped around the bending section 32. Typically, the polymeric jacket 74 is comprised of polyvinyl chloride or urethane, although other materials may be used as well. The structure of the vertebra 39 covered by the metal braid 72 and that assembly overlaid by the polymeric jacket 74 constitutes the completed bending section of the insertion tube, as shown in FIG. 1A.

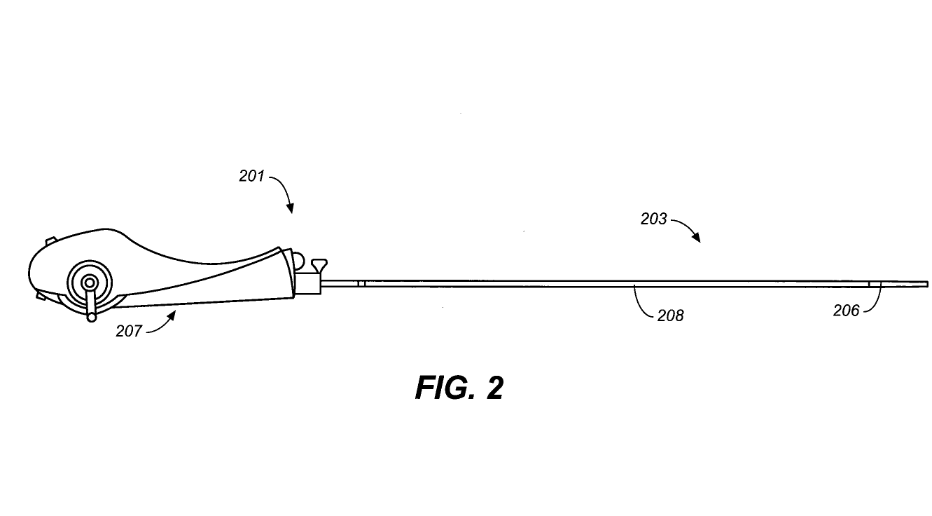

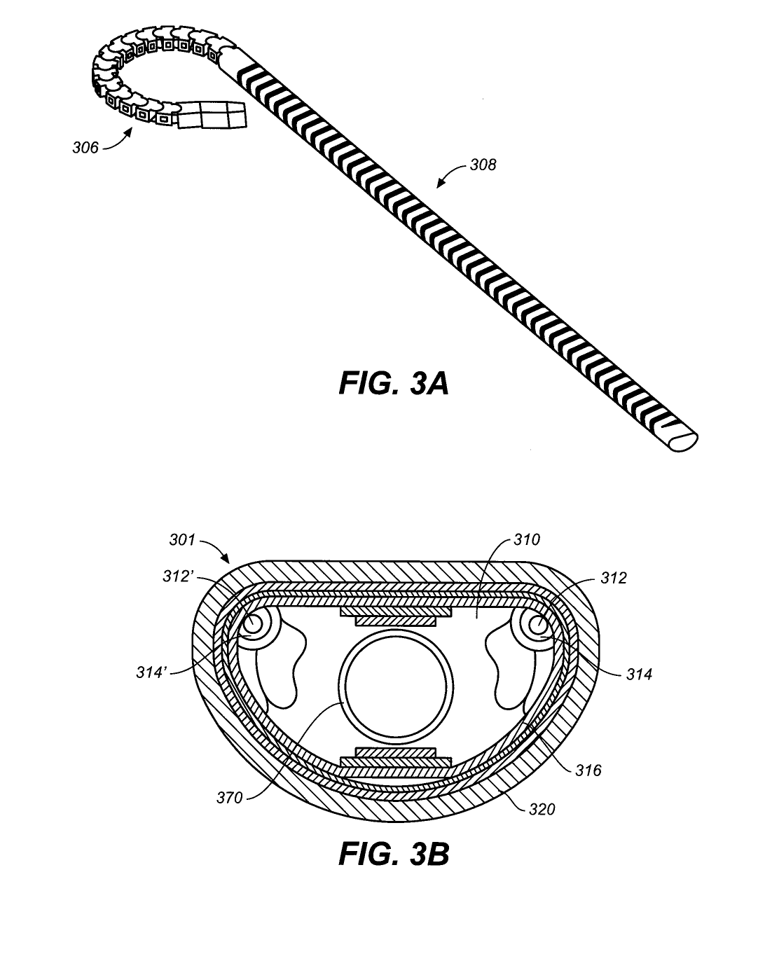

FIG. 2 shows one variation of an endoscope 201 having a non-circular outer perimeter of the insertion tube 203 cross-section. In FIG. 2, the outer perimeter of the insertion tube 203 is approximately “D”-shaped, as shown in FIG. 3B in cross-section, described below. The insertion tube 203 may include a bending portion 206 and an elongate portion 208. In this example, the bending portion 206 is located at the distal end of the insertion tube, adjacent to the elongate portion 208. Different lengths of elongate portions and bending portions may be used. In some variations, the elongate portion overlaps with the bending portion. The endoscope shown in FIG. 2 includes a handle 207 with various controls that may be used to control the tip of the endoscope (e.g., the bending region 206).

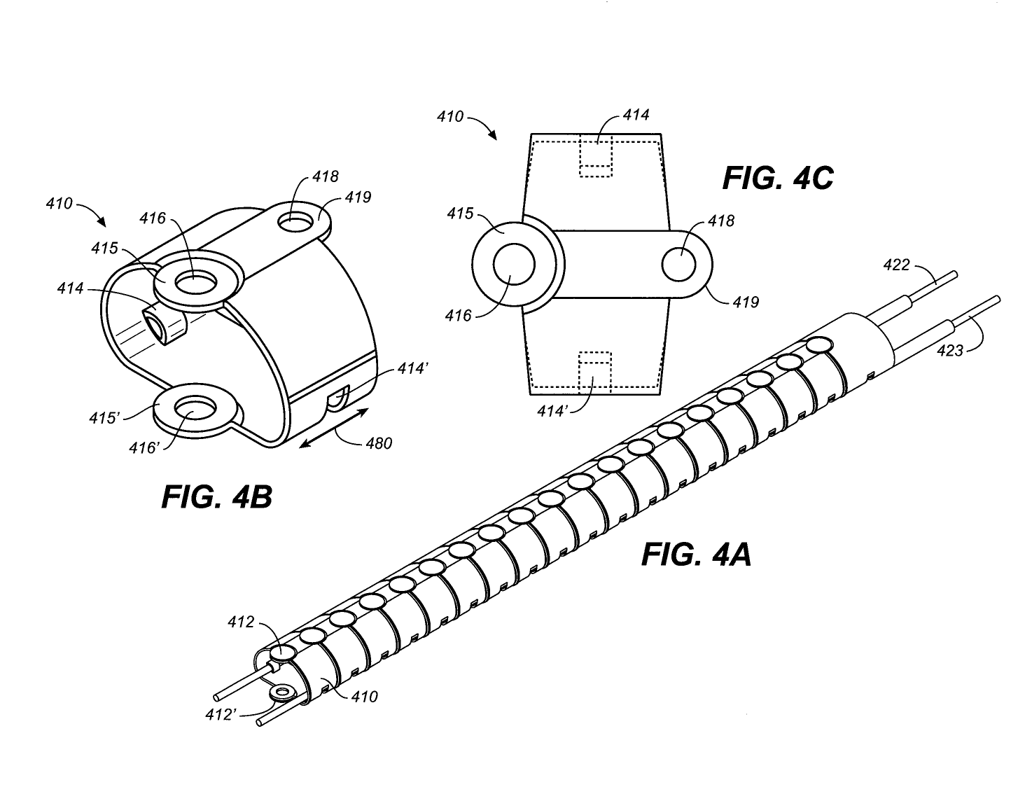

FIGS. 3A through 4C further illustrate the variation of the conforming endoscope shown in FIG. 2. For example, FIG. 3A shows a partial perspective view of the insertion tube (i.e., the insertion tube 203 shown in FIG. 2) and bending section. In this example, the bending portion 306 and the elongate portion 308 are partially made of vertebrae that are joined together. FIG. 3B shows a cross-section through the insertion tube, and shows that the vertebrae are approximately D-shaped in cross-section. In FIG. 3A, the vertebrae making up the bending portion 306 are joined so that they may bend or flex within one plane (i.e., the direction horizontal to the flat side of the D-shape), shown in FIG. 4A and described below. In some variations, adjacent vertebrae are not directly joined or hinged, but may be connected via a separate hinge element or via a cable. The vertebrae making up the elongate portion 308 may be similarly joined. In some variations, the elongate portion is separate from the bending portion, and the elongate portion does not include vertebrae. For example, the elongate region may be a flexible tube or member (e.g., a cut or notched hypotube). The insertion tube (including both the bending and elongate portions) may have sufficient column strength so that they can be inserted by pushing from the proximal end of the device.

The insertion section may be prepared for insertion into a subject's body by preparing the outer surface of the insertion section so that it may contact the subject with minimal risk of contamination and damage to the subject. For example, the structure shown in the partial view of FIG. 3A may be covered with one or more jackets or sheaths. A jacket or sheath may provide smoother, more patient-friendly outer surface for contacting the subject. A jacket or sheath may be made of an appropriately flexible material, as known in the art. The jacket and/or sheath may also be made of a sterilizable material, and/or may be disposable, negating the need to perform high level disinfection of the endoscope between uses on different patients.

The cross-section of the insertion section shown in FIG. 3B, shows that the insertion section has a non-circular, D-shaped outer perimeter cross-section 301. The outer (patient-contacting) surface in this example includes an outer sheath 320. The inner region includes a vertebra 310 having two apertures 314, 314′ through which control wires 312, 312′ (respectively) pass. The cross-sectional shape of the vertebra 310 reflects the shape of the outer perimeter 301 of the insertion section. The vertebra 310 surrounds a central region 370 in which one or more channels or passages may pass. For example, biopsy channels, air/water channels, optic channels, light channels, power channels, or the like. In some variations, some or all of these channels may be included. In some variations, these channels may be included outside of the bounds of the vertebra (e.g., between the vertebra and the sheath, or as part of the outer sheath or an inner sheath or jacket).

In FIG. 3B the vertebra 310 includes an inner jacket 316. The inner jacket may comprise any appropriate material. For example, the jacket may be made of the same material as a sheath. In some variations, the jacket is a polymeric material. Some variations of the devices described herein do not include a jacket. In some variations, an additional surrounding layer (e.g., a braided layer, etc.) may be included.

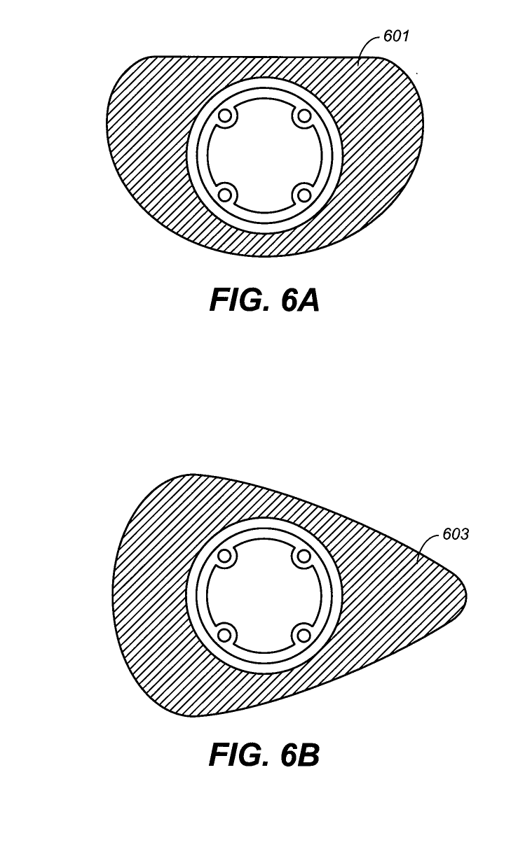

The vertebrae shown and described above may be made of any appropriate material, including (but not limited to) metals, plastics, ceramics, or combinations thereof. Although the example shown herein includes adjacent vertebrae having substantially similar structures, different vertebrae (e.g., vertebrae having different shapes or cross-sectional profiles) may be included. In addition, in some variations the vertebra does not have the same cross-sectional profile (e.g., non-circular profile) as the outer perimeter profile of the insertion tube that is to be inserted into a subject. For example, the vertebrae of the bending portion may be circular in cross-sectional profile. In such variations, a sheath (e.g., an outer sheath) may provide the non-circular cross-section. An example of this is shown in FIG. 6 for an endoscope having a D-shaped outer perimeter.

FIG. 4A shows a partial perspective view of a bending portion similar to the bending portion described above for FIG. 3A in which adjacent vertebrae 410 have been flexibly joined by opposed hinged regions 412, 412′. In this example, identical vertebrae 410 are connected, and two control wires 422, 423 are threaded through them. The vertebrae 410 are arranged so that they may bend in two directions. As described above, vertebrae may be configured so that they may bend in any appropriate combination of directions, and additional control wires may be used.

FIGS. 4B and 4C show side perspective and top views of a D-shaped vertebra 410. The D-shaped vertebra has a thickness (480) which may be selected from any appropriate range. The vertebra in this example is a tubular structure (have a D-shaped profile). The side wall(s) of the vertebra 410 includes two apertures 414, 414′ through which the control wires 423 (not shown in FIG. 4B or 4C) may run. The apertures 414, 414′ in this example are formed by making parallel cuts in the sidewall(s) and indenting the cut region.

In FIGS. 4B and 4C, the hinged regions joining adjacent vertebrae are formed from structures at the top and bottom of each vertebra that join. In FIG. 4B, the distal end (when connected to an endoscope) of the vertebra 410 has two slightly recessed disk-shaped regions 415, 415′ on the upper and lower surface. The upper disk-shaped region 415 on the distal end of a vertebra mates with an upper tab-like region 419 on the proximal side of the adjacent vertebra. Similarly, a lower disk-shaped region 415′ on the distal end of a vertebra mates with a lower tab region on the proximal end of an adjacent vertebra. Both the disk-shaped regions 415, 415′ and the tab regions 419 are shown having a hole 416, 416′ and 418. The holes in the disk-shaped regions 416 may be lined up with the holes in the tab regions 418, and a hinge pin (not shown) may be inserted to secure the disk and tab hinge regions together. The proximal and distal edges of the vertebrae may be tapered so that they do not interfere with the edges of an adjacent vertebra, and they may therefore permit the hinged vertebrae to bend in at least one plane, as described above.

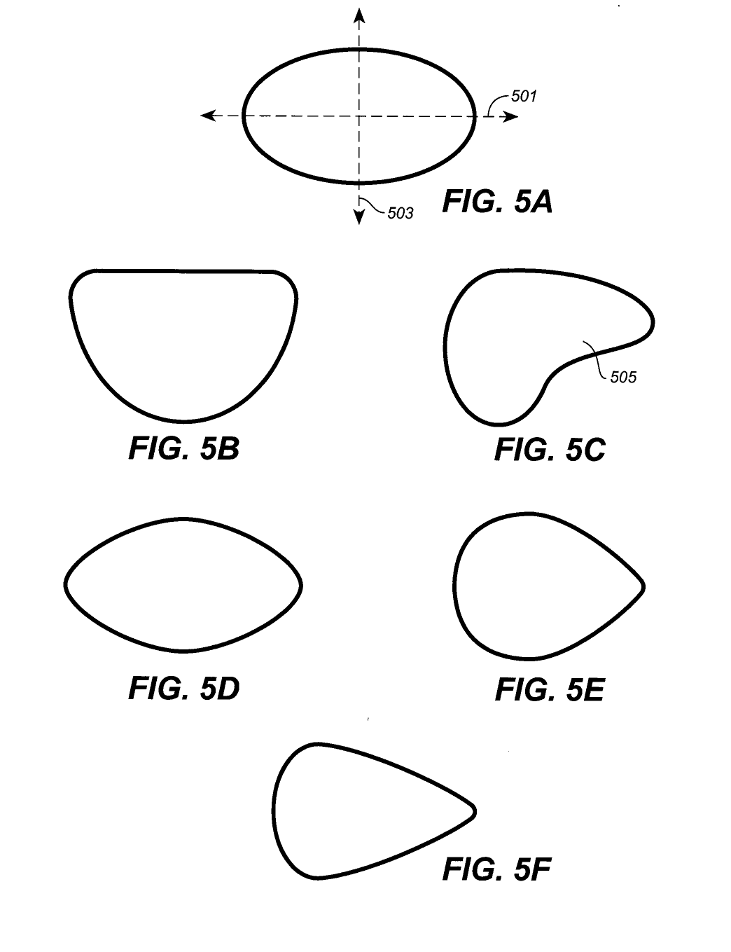

Although the insertion region of the endoscope described above shows devices having outer perimeters that are D-shaped, any appropriate non-circular outer perimeter shape may be used for the insertion region. In particular, outer perimeter cross-sectional shapes that provide one or more narrower protrusion along the length of the insertion region (e.g., a rim, ledge, etc.) and that match the shapes of the body region into which the endoscope will be placed may be useful. FIGS. 5A to 5F illustrate some variations of non-circular outer perimeter cross-sections that may be used with any of the devices described herein.

FIG. 5A shows a cross-section of an outer perimeter of an insertion region of an endoscope that is approximately elliptical. In FIG. 5A, a major axis 401 and a minor axis 403 are also shown on the non-circular (elliptical) cross-section. The major axis is the long axis (e.g., the longest straight line between opposite points around the outer perimeter of the cross-section). The minor axis is perpendicular to the major axis. In this elliptical cross-section, the minor axis is the short axis (the shortest straight line between opposite points around the perimeter). The elliptical cross-section is symmetrical about the major axis 401 and is also symmetrical about the minor axis 403. Major and minor axes may be identified on any of the outer perimeter cross-sections illustrated in FIGS. 5B-5F as well. In some variations, the outer perimeter of the insertion tube is not symmetric about either (or both) the major and minor axes. For example, FIGS. 5E and 5F are symmetric about the major axis, but not the minor axis. FIG. 5B is symmetric about the minor axis but not the major axis. FIG. 5C is not symmetric about either the major or minor axis, and FIG. 5D, like FIG. 5A, is symmetric about both the major and minor axes.

FIG. 5B shows an outer perimeter for an insertion tube similar to that described in FIG. 3B, above. FIG. 5C shows an outer perimeter for an insertion tube that is relatively asymmetric, and includes a protruding region 505. FIG. 5D shows a semi-elliptical cross-section of an outer perimeter of an insertion tube, in which the region of the device along the long axis are tapered. FIGS. 5E and 5F both show teardrop-shaped outer-perimeter cross-sections having major and minor axes of different lengths.

The insertion tube outer perimeter cross-sections shown in FIGS. 5A-5F are variations of the cross-sectional shapes for the outer surface of the insertion region of the endoscopes or endoscope systems described herein. Thus, they represent the shape of the outer surfaces of the insertion region of an endoscope when a cross-section is taken perpendicular to the long axis of the insertion region. The cross-section may be taken at any point along the length of the long axis of the insertion region. In some variations, the insertion region may have different outer perimeter cross-sections at different points along its length. For example, at least one continuous portion of the elongate portion of the insertion tube may have a non-circular cross-section for the outer perimeter. In some variations, the bending portion of the insertion tube may have the same (or a different) non-circular cross-section for the outer perimeter.

The major and minor axes may be any appropriate length. For example, in some variations, the major axis of the cross-section through the outer perimeter of the insertion tube is between about 2 mm and about 5 mm. In some variations, the major axis is less than about 4.3 mm. In some variations, the length of the minor axis of the cross-section through the outer perimeter of the insertion tube is less than about 3 mm. In some variations, the minor axis is less than about 2.8 mm.

Although FIGS. 5A to 5F do not show the internal details of the cross-section though the insertion tube, similar internal features to those shown in FIG. 3B (e.g., vertebra, wires, channels, sheaths, jackets, etc.) may be found within each of the insertion tubes shown in cross-section in FIGS. 5A to 5F. FIGS. 5A to 5F have been simplified to show only the outer perimeter.

In operation, the non-circular outer perimeters shown in FIGS. 5A-5F may allow an insertion tube to fit within and conform to the lumen, orifice or other body region into which it has been inserted. These devices may be used within any appropriate body region, including (but not limited to) esophagus, stomach, small and large bowel, intestine, bile ducts, large and small respiratory airways, nasal cavities, urethras, fallopian tubes, etc. Many of these body regions have non-circular internal lumen shapes. Thus, it may be beneficial to use an appropriately configured endoscope (e.g., a conforming endoscope) with each of the body regions described. The insertion tubes having non-circular cross-sections may fit into body regions that insertion tubes having approximately circular outer diameter cross-sections, having the same cross-sectional area, could not fit into. In this way, the space inside the body region utilized by the endoscope is not limited to the minor dimension of the body region. Thus, the available space inside the body region may be more fully utilized. Looking at the nasal passageway as one non-limiting example, most subjects' nasal passageways have a non-circular (e.g., lobular) cross-section. If an endoscope having an outer perimeter whose geometry is matched to the inner perimeter cross-section of a body region, the insertion tube may fit the subject's body region better, and the insertion tube may also have a larger overall cross-sectional area than an endoscope having an approximately circular cross-section that is inserted into the same body region. This may permit additional or larger channels to be used with the endoscope. These scopes may also be more comfortable to use.

As mentioned above, endoscopes may be tailored for a pre-determined body region. For example, any of the endoscopes (or principles for making endoscopes) described herein may be adapted to be a nasal endoscope for use in a subject's nasal passageway. Thus, the outer perimeter of the insertion section of an endoscope may be configured to match (approximately) the shape of the nasal passageway into which it is intended to be inserted. The general shapes and dimensions of a body region (such as the nasal passageway) are well characterized, and may be found by reference to the literature and standard (or size/age/population-specific) references (e.g., Frank Netter, Atlas of Human Anatomy, 4th Edition, Saunders (Jun. 26, 2006); Henry F. R. S. Gray and R. A. Bolam, Gray's Anatomy, Merchant Book Company Limited, New Ed. edition (Mar. 30, 2003)). In some cases, an endoscope may be matched to the geometry of the target body region based on average or mean population characteristics. In some variations, an endoscope may be matched to the geometry of a specific subject or subset of subjects.

Similarly, urethral endoscopes, or any other body region-specific endoscopes may be formed as described herein. The overall shape and dimension of these endoscopes (and particularly the insertion tubes of the endoscopes) may be determined based on both the size and shape of the body region, as well as the orifice (or other access way) into the region of the body into which the endoscope will be inserted. For example, the dimensions of the insertion region of a nasal endoscope may be in part determined by the nostril opening.

FIGS. 6A and 6B show cross-sections through the different insertion tube portions of endoscopes systems as described herein. As mentioned briefly above, the outer (i.e., subject-contacting) surface of an endoscope system may include a sheath. In some variations, the sheath provides the non-circular cross-sectional shape of the insertion region. FIG. 6A shows a cross-section through the insertion tube of an endoscope having a D-shape. In this example, the outer perimeter of the D-shaped insertion tube is defined primarily by the sheath 601. The sheath surrounds an inner region including a plurality of vertebrae 610. In this example, the vertebrae have a circular cross-section, and the sheath acts as an adapter to achieve the D-shaped perimeter. In other variations, the vertebrae (part of either, or both, the bending portion and the elongate portion) may also have a non-circular cross-section. Similarly, FIG. 6B shows a cross-section through an insertion tube of an endoscope having a generally triangular or wedge-shape, in which the adapter or sheath 603 determines the triangular outer perimeter shape. If the insertion tube (including the sheath) is non-circular, and bending portion is circular of size to fit within insertion portion, one may want to make use of the space that would be within the non-circular cross-section of the insertion portion but outside of the circular portion of the bending portion (e.g., for light delivery, etc.).

All publications, patents, and patent applications cited herein are hereby incorporated by reference in their entirety for all purposes to the same extent as if each individual publication, patent, or patent application were specifically and individually indicated to be so incorporated by reference. Although the foregoing endoscopes have been described in some detail by way of illustration and example for purposes of clarity of understanding, it will be readily apparent to those of ordinary skill in the art, in light of the description herein provided, that certain changes and modifications may be made thereto without departing from the spirit and scope of the appended claims.

1. An endoscope for performing a diagnostic or therapeutic procedure comprising:
a) an elongate portion, non-round in cross-section, for insertion into an organ or passageway of the body; and
b) a bending portion,
wherein the cross-section of the elongate portion has a major axis and a minor axis and wherein the elongate portion is configured to accommodate the geometry of the organ or passageway, and the bending portion comprises a plurality of vertebrae.

2. The endoscope of claim 1, wherein the elongate portion is asymmetric about the minor axis, the major axis, or both the minor and major axis.

3. The endoscope of claim 1 wherein the plurality of vertebrae are non-round in cross-section.

4. The endoscope of claim 1 wherein the elongate portion is D-shaped in cross-section.

5. The endoscope of claim 1 wherein the plurality of vertebrae are D-shaped in cross-section.

6. The endoscope of claim 1 wherein the organ is a urologic organ.

7. The endoscope of claim 1 wherein the urologic organ is the ureter.

8. The endoscope of claim 1 wherein the urologic organ is the urethra.

9. The endoscope of claim 1 wherein the passageway is a respiratory passageway.

10. The endoscope of claim 1 wherein the respiratory passageway is a nasal passageway.

11. The endoscope of claim 1 further comprising a sheath having a proximal end, a distal end, and a sheath lumen extending from the proximal end to the distal end configured for slidable advancement of the elongate portion.

12. The endoscope of claim 11 wherein the sheath is configured to have variable stiffness between the proximal end and distal end.

13. The endoscope of claim 11 wherein the sheath lumen is non-round in cross-section.

14. The endoscope of claim 11 wherein the sheath lumen is D-shaped in cross-section.

15. The endoscope of claim 1 wherein the elongate portion comprises a shaft having a proximal end, a distal end, and a length therebetween of variable stiffness.

16. The endoscope of claim 1 wherein the bending portion is part of the elongate portion.

17. The endoscope of claim 1 wherein the bending portion rotates about its minor axis.

18. The endoscope of claim 1 wherein the bending portion rotates about its major axis.

19. The endoscope of claim 1 wherein the length of the minor axis is about 3 mm or less.

20. The endoscope of claim 1 wherein the length of the major axis is about 4.3 mm or less.

21. An endoscope for performing a diagnostic or therapeutic procedure comprising:
a) an elongate portion, non-circular in cross-section, for insertion into a portion of a body; and
b) a bending portion,
wherein the cross-section of the elongate portion and/or the bending portion has a major axis and a minor axis, and wherein the elongate portion is configured to accommodate the geometry of the portion of a body, and the bending portion comprises a plurality of vertebrae.

22. An endoscope for performing a diagnostic or therapeutic procedure comprising:
a) an elongate portion, having a non-circular outer perimeter in cross-section, for insertion into an organ or passageway of the body; and
b) a bending portion,
wherein the outer perimeter cross-section comprises a protuberance, wherein the protuberance is configured to conform to the geometry of the organ or passageway, and the bending portion comprises a plurality of vertebrae.

23. A system for performing a diagnostic or therapeutic procedure, the system comprising:
an endoscope having a an elongate portion, non-circular in cross-section, for insertion into an organ or passageway of the body; and a bending portion, wherein the cross-section through the elongate portion has a major axis and a minor axis and wherein the elongate portion is configured to accommodate the geometry of the organ or passageway, and the bending portion comprises a plurality of vertebrae;
an outer sheath configured to fit over the endoscope so that the outer perimeter of a cross-section through the endoscope and the outer sheath is a non-round cross-section.

24. A method of performing a diagnostic or therapeutic procedure, the method comprising:
inserting the insertion portion of an endoscope system into a body lumen, wherein the insertion portion of the endoscope system has a non-circular outer perimeter in cross-section, and wherein the inner perimeter of the body lumen has a non-circular cross-section.

25. The method of claim 24, wherein the non-circular outer perimeter cross-section comprises a D-shaped cross-section.

26. The method of claim 24, wherein the non-circular outer perimeter cross-section comprises an asymmetric cross-section.

27. The method of claim 24, further comprising:
preparing the insertion portion of the endoscope system by placing a sheath over the endoscope prior to inserting the insertion portion of the endoscope into the body lumen.

28. An endoscope for performing a diagnostic or therapeutic procedure comprising:
an insertion portion having a non-circular outer perimeter in cross-section, for insertion into an organ or passageway of the body while maintaining the non-circular outer perimeter; and
a bending portion,
wherein the outer perimeter cross-section comprises a protuberance, wherein the protuberance is configured to conform to the geometry of the organ or passageway, and wherein the bending portion comprises a plurality of vertebrae.

29. The endoscope of claim 28, wherein the outer perimeter cross-section changes over the length of the insertion portion.

30. The endoscope of claim 29, wherein the outer perimeter cross-section of the insertion portion is larger towards the proximal end of the insertion portion than the distal end of the insertion portion.
